(19) United States
(12) Patent Application Publication
(10) Pub. No.: US 2024/0316306 A1
(43) Pub. Date: Sep. 26, 2024

(54) PORTABLE OXYGEN CONCENTRATOR WITH UPSTREAM FILTER UNIT

(71) Applicant: AXNAR MED TECH GmbH, Hamburg (DE)

(72) Inventor: Jochen Hölscher, Hamburg (DE)

(21) Appl. No.: 18/613,906

(22) Filed: Mar. 22, 2024

(30) Foreign Application Priority Data
Mar. 24, 2023 (EP) 23164019.4

(51) Int. Cl. A61M 16/10; B01D 53/047

(52) U.S. Cl. CPC A61M 16/101; A61M 16/105; B01D 53/0476; B01D 2256/12; B01D 2259/40007; B01D 2259/4541

(57) ABSTRACT

The object of the disclosure is a portable oxygen supply device including an oxygen concentrator, at least one air inlet through which ambient air is transported into the oxygen supply device, one oxygen outlet through which enriched oxygen is transported from the oxygen supply device, one exhaust air outlet through which exhaust air is transported from the oxygen supply device, a gas line system which is fluidically connected to the at least one air inlet, the outlet and the oxygen concentrator, a power source, at least one pump and a housing, wherein all components of the oxygen supply device are arranged in or on the housing and wherein the air inlet has an intake nozzle through which the ambient air is sucked into the oxygen supply device. The oxygen supply device has a filter unit.

### CROSS REFERENCE TO RELATED APPLICATION

The present application claims priority to Application Ser. No. 23164019.4, filed in the European Patent Office on Mar. 24, 2023, which is expressly incorporated herein in its entirety by reference thereto.

### TECHNICAL FIELD

The present disclosure relates to a portable oxygen supply device for supplying a patient and a ventilation system.

### BACKGROUND

Oxygen from bottles or from an oxygen generator, such as an oxygen concentrator, is used to supply oxygen to patients, in particular in emergency care or in the domestic care of chronically ill patients. An oxygen concentrator sucks in air from its immediate environment via a pump. The sucked in air flows through a plurality of filters to remove dirt or germs. The purified air is then compressed. The nitrogen contained in the air is absorbed by an adsorber, the so-called molecular sieve, and separated from the remaining air. In this way the oxygen in the delivered air increases from >21% to up to 96%.

In the final phase, the filtered nitrogen is removed from the molecular sieves in the oxygen concentrator and the concentrated oxygen is delivered to the patient. After the adsorbers have been regenerated, the process starts again.

In order to ensure the continuous and constant supply of concentrated oxygen, oxygen concentrators work with two of the systems described above in parallel, so that one system is always in the suction, concentration and regeneration phase, thus ensuring the constant supply of concentrated oxygen to the patient.

Conventional oxygen concentrators are usually used in clinical or domestic areas and thus in a relatively clean environment. They therefore only have filters which protect against coarse impurities, such as HEPA filters. A HEPA filter, abbreviation for the English term “High Efficiency Particulate Air”, is a mechanical filter which filters suspended particles from the air.

During pre-hospital emergency medical care, the scene of the emergency may be contaminated with nuclear, biological or chemical substances. As a result, possible substances are passed into the device and then directly to the patient. This means that the device cannot be reused after being used and the patient is not being protected.

Modern oxygen concentrators are only designed for use in “safe areas” such as domestic and hospital environments. This means that only HEPA filters are used, which do not provide protection against contamination of the patient or the device, but rather are intended to protect against damage to the pump.

WO 03/015899 A1 describes an oxygen concentrator based on a vacuum pressure swing adsorption (VPSA) system having a filter. The filter is arranged upstream of the compressor so that particles are filtered out upstream of the compressor and the compressor is protected from mechanical damage.

It is therefore the object of the present disclosure to provide an oxygen supply device which overcomes the above disadvantages and allows safe use even in prehospital emergency medicine.

### SUMMARY

The portable or stationary oxygen supply device according to the disclosure for supplying a patient includes:
an oxygen concentrator,
at least one air inlet through which ambient air is transported into the oxygen supply device,
an intake nozzle fluidically connected to the at least one air inlet through which the ambient air is sucked into the oxygen supply device,
at least one oxygen outlet through which enriched oxygen is transported from the oxygen supply device,
at least one exhaust air outlet through which exhaust air is transported from the oxygen supply device,
a gas line system which is fluidically connected to the at least one air inlet, the oxygen outlet, the exhaust air outlet and the oxygen concentrator,
a power source,
at least one pump,
a housing, wherein all components of the oxygen supply device are arranged in the housing or on the housing, and
a filter unit, the filter unit is either fastened directly to the intake nozzle or fastened to the intake nozzle with an adapter upstream of the air inlet, and the filter unit is selected from an NBC filter, combination filter and particle filter.

The oxygen supply device may be a mobile device which has at least one battery unit or one accumulator as a power source and has a shock-resistant housing.

The filter unit may be a filter unit for a respirator mask/NBC protective mask, in particular an NBC filter unit, a particle filter unit or a combination filter unit.

The intake nozzle may include a threaded connection arranged on the side of the air inlet facing the ambient air.

The threaded connection may be a threaded connection Rd40 according to the standard EN 148 Part 1.

The oxygen supply device may further include a detection mechanism arranged in the intake nozzle, wherein the detection mechanism detects whether a filter unit is inserted and, if applicable, additionally detects which type of filter is inserted.

The detection mechanism may be a mechanical pressure switch in the thread of the intake nozzle.

The detection mechanism may be connected to a control unit and the control unit controls the output of the oxygen concentrator and, if applicable, is connected to an information unit which provides the user with information on the presence of a filter unit and/or the changed output of the oxygen concentrator.

The oxygen concentrator may be a pressure swing adsorption (PSA) system or a vacuum pressure swing adsorption (VPSA) system and has one or a plurality of adsorber columns.

The oxygen supply device may have one or a plurality of sensors in the housing, which may be connected to the gas line system.

An oxygen supply system according to the disclosure includes an oxygen supply device and a vehicle-compatible device holder with a securing device.

The air inlet may have an intake nozzle through which the ambient air is sucked into the oxygen supply device. The oxygen supply device has a filter unit so that ambient air first flows through the filter unit and is filtered before entering the oxygen supply device. The filter unit is fastened upstream of the air inlet. It is either fastened directly to the intake nozzle itself or fastened to the intake nozzle with an adapter. The filter unit is connected in a gas-tight manner to the air inlet. The filter unit is selected from an NBC filter, combination filter and particle filter.

In one embodiment, the oxygen supply device is a mobile or semi-stationary device which has at least one battery unit or at least one accumulator as a power source and has a shock-resistant housing. Semi-stationary is understood to mean, for example, a larger device in a transport box or a transport container, which device can be transported to the place of use with auxiliary means. Alternatively, the oxygen supply device can also be operated with conventional mains power, e.g. via a generator.

The filter unit is preferably formed as a filter unit for a respirator mask, also known as an NBC protective mask. In particular, the filter unit is an NBC filter unit for a respirator mask, a particle filter unit for a respirator mask or a combination filter unit for a respirator mask. The filter unit can additionally have a HEPA filter.

An NBC filter unit, also known in German as an ABC filter unit, protects against chemical warfare agents, toxic industrial chemicals, microorganisms and radioactive particles. The label NBC stands for nuclear, biological and chemical. The European standard for these filters is EN 14386 for industrial filters.

In one embodiment, the intake nozzle has a threaded connection arranged on the side of the air inlet facing the ambient air. The threaded connection is preferably a threaded connection Rd40, i.e. a round threaded connection with 40 mm× 1/7 inch according to the standard EN 148 Part 1).

By designing the intake nozzle according to the standard threaded connection Rd40 according to EN 148 Part 1) or the NATO standard STAGNAG 4155 and with an inner diameter of 22 mm, commercially available NBC filters and commercially available medical filters for respiratory filters, possibly supplemented by additional adapters, can be attached to the oxygen supply device, making it possible to protect the patient and the device. The use of standard filters for respiratory masks also ensures good availability of filter units in difficult areas of operation, such as war or crisis zones or during emergency operations, since already available filter units from respiratory masks can be used.

A detection mechanism can be arranged in the intake nozzle or air inlet, wherein the detection mechanism detects whether a filter unit is inserted at all and, if applicable, additionally detects which type of filter is inserted. The detection mechanism can have one or a plurality of detection units.

The detection mechanism can have a mechanical pressure switch as a detection unit in the thread of the intake nozzle.

In one embodiment, the oxygen supply device has a control unit. The detection mechanism is connected to the control unit. The control unit controls the output of the oxygen concentrator. If applicable, the control unit is connected to an information unit which provides the user with information on the presence of a filter unit and/or the changed output, e.g. via a display or another display device.

The oxygen concentrator can be a pressure swing adsorption (PSA) system or a vacuum pressure swing adsorption (VPSA) system and have one or a plurality of adsorber columns. The adsorbers are preferably zeolites. This is a special material for adsorbing nitrogen, whereby an oxygen-rich gas is obtained.

The oxygen supply device may have one or a plurality of sensors in the housing, which are preferably connected to the gas line system. The sensors can be, for example, pressure sensors, temperature sensors, gas sensors for determining a gas concentration, flow sensors, sensors for detecting the power supply or humidity sensors.

The oxygen supply device may have other components, such as silencers, humidifiers or display units, such as displays.

Another object of the disclosure is an oxygen supply system having an oxygen supply device according to the disclosure and a vehicle-compatible device holder with a securing device. The holder for the vehicle is preferably a device holder for a land vehicle, an aircraft or a watercraft, preferably for an ambulance, a rescue helicopter or an ambulance aircraft.

Modern oxygen concentrators are only designed for use in “safe areas” such as domestic and hospital environments. The HEPA filters used do not offer any protection against contamination.

The filter unit protects the oxygen supply device according to the disclosure from contamination or damage by nuclear, biological or chemical impurities or substances or large particles.

The oxygen supply device according to the disclosure is particularly suitable for use with enormous exposure to sand, such as when used in a military context in the desert or dry areas or with open doors in a helicopter. During take-off and landing in a helicopter, a “downwash” occurs. Massive swirling of sand can occur during these operations. In conventional devices, this sand is then sucked in in the suction protection in the oxygen concentrator and can destroy internal components. In the oxygen supply device according to the disclosure, the sand is trapped in the filter unit and the internal components are thus protected. After use, the dirty filter unit can be replaced and the device is ready to be reused.

The oxygen supply device is also protected against contamination when used in infectious environments, e.g. in hospitals. Conventionally, the room air is sucked into the device, wherein pathogenic germs are not stopped by the standard HEPA filter, thus allowing cross-contamination with other patients. In the oxygen supply device according to the disclosure, the microorganisms are trapped in the filter unit so that other patients and staff are protected from contamination. Here too, the contaminated filter unit can be replaced after use and the device is ready to be reused. The use of upstream filter units results in an increase in resistance at the air inlet of the oxygen concentrator. To compensate for the increase in pressure, the output of the vacuum pump and the pressure pump is adjusted. In addition, the user can be informed via an information unit about the changed output parameters, such as shortened battery life, reduced acid concentration or reduced throughput. By detecting the filter unit via the detection mechanism in the screw thread, the regulation can be started and stopped automatically with a control unit and a filter type detection can be carried out without the user having to intervene.

Further features, details and advantages of the disclosure are apparent from the wording of the claims and from the following description of embodiment examples with reference to the drawings. The Figures show:

### BRIEF DESCRIPTION OF THE DRAWINGS

FIG. 1 shows a schematic drawing of an oxygen supply device according to the disclosure;

FIG. 2 shows a top view of the air inlet without mounted filter unit; and

FIG. 3 shows a flow diagram for the operation of an oxygen supply device according to the disclosure.

### DETAILED DESCRIPTION

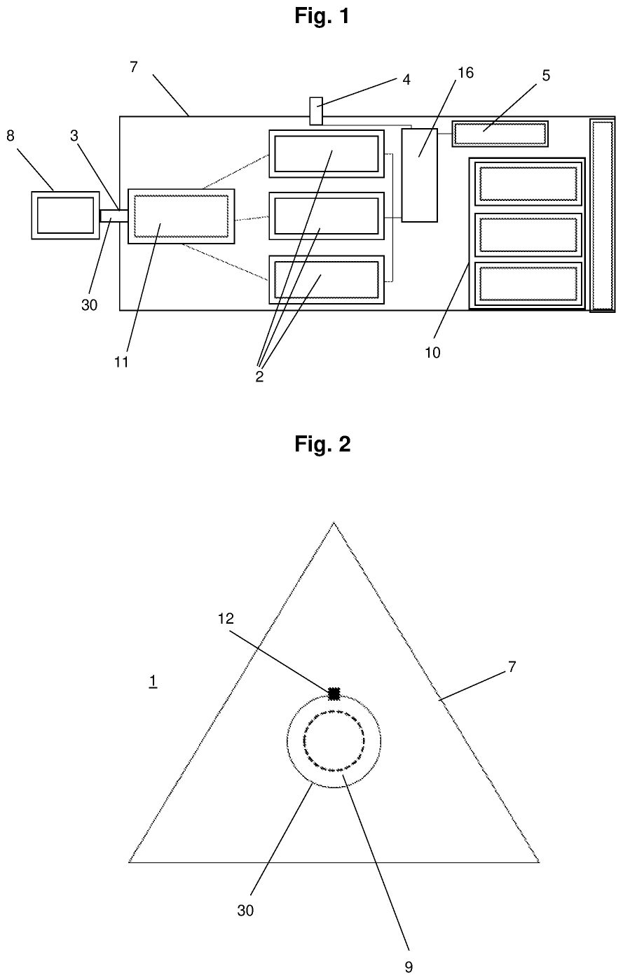

FIG. 1 shows a schematic structure of an oxygen supply device according to the disclosure. An oxygen concentrator 2, a power source 10 and two pumps 11 and 16 are arranged in a housing 7. On the housing there is an air inlet 3 which leads through the housing wall. The air inlet has an intake nozzle 30 on the outside of the housing. A filter unit 8 is arranged upstream of the intake nozzle and is connected to the intake nozzle 30 in a gas-tight manner. The exhaust air outlet 5 also leads through the housing 7 and enables exhaust air to be transported from the oxygen supply device. The oxygen outlet 4 is connected to a tube which leads to the patient.

FIG. 2 shows a top view of the side of the oxygen supply device 1 according to the disclosure with air inlet 3 without mounted filter device. The air inlet leads from the outside through the housing 7 into the inside of the oxygen supply device. The air inlet 3 is formed as an intake nozzle 30 on the outside of the housing 7. The intake nozzle 30 has a screw thread 9 on the inside. A switch 12 is arranged in the screw thread 9. The switch 12 serves as a detection mechanism for a filter unit (not shown).

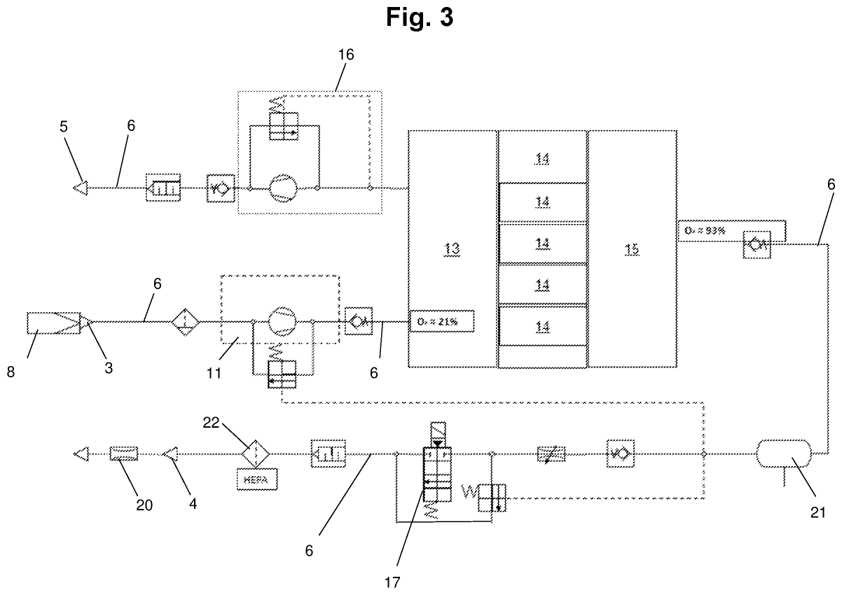

FIG. 3 shows a flow diagram for an operating variant of an oxygen supply device 1 according to the disclosure with VPSA system. Ambient air is sucked through a filter unit 8 via the air inlet 3 into the gas line system 6 to a first pressure pump 11. The pressure pump 11 transports the ambient air through the gas line system 6 to a first distributor 13, which has a plurality of valves. The compressed ambient air is fed from the distributor 13 to the adsorber units 14 so that the air flows through the adsorber units. Nitrogen and other components from the ambient air are adsorbed in the adsorber unit 14, so that a gas with an increased concentrated oxygen content is produced.

The gas with concentrated oxygen content is fed from the adsorber unit 14 via a second distributor 15, which has a plurality of valves, and via a two-way valve 17 to the oxygen outlet 4 and from there via a tube 20 to the patient. After a while, the adsorber unit is largely saturated. At this point, the valves in the first distributor 13 and in the second distributor 15 are switched so that the gas line to the oxygen outlet 4 is closed and the connection to the exhaust air outlet 5 is opened for the exhaust air. The adsorbed gas is desorbed again. Alternately loaded and unloaded adsorbers ensure continuous operation. In the VPSA system design, the oxygen generation device has a vacuum pump 16 in fluidic connection with the first distributor 13. The exhaust air is sucked from the adsorber unit 14 via the first distributor 13 into the vacuum pump 16 and transported from there to the exhaust air outlet 5. The oxygen supply device can additionally be equipped with an oxygen buffer tank 21 in which excess oxygen is stored. For additional filtering of the air, a HEPA filter 22 is arranged upstream of the oxygen outlet 4 in the gas line system 6.

The disclosure is not limited to one of the embodiments described above, but can be modified in a variety of ways.

All features and advantages resulting from the claims, the description and the drawings, including design details, spatial arrangements and method steps, can be essential to the disclosure both individually and in various combinations.

1. A portable oxygen supply device for supplying a patient, comprising:
an oxygen concentrator;
at least one air inlet through which ambient air is transported into the oxygen supply device;
an intake nozzle fluidically connected to the at least one air inlet through which the ambient air is sucked into the oxygen supply device;
at least one oxygen outlet through which enriched oxygen is transported from the oxygen supply device;
at least one exhaust air outlet through which exhaust air is transported from the oxygen supply device;
a gas line system which is fluidically connected to the at least one air inlet, the outlet and the oxygen concentrator;
a power source;
at least one pump;
a housing, wherein all components of the oxygen supply device are arranged in or on the housing; and
a filter unit, the filter unit is either fastened directly to the intake nozzle or fastened to the intake nozzle with an adapter upstream of the air inlet, and the filter unit is selected from an NBC filter, combination filter and particle filter.

2. The oxygen supply device according to claim 1, wherein the oxygen supply device is a mobile device which has at least one battery unit or one accumulator as a power source and has a shock-resistant housing.

3. The oxygen supply device according to claim 1, wherein the filter unit is a filter unit for a respirator mask/NBC protective mask, in particular an NBC filter unit, a particle filter unit or a combination filter unit.

4. The oxygen supply device according to claim 1, wherein the intake nozzle includes a threaded connection arranged on a side of the air inlet facing the ambient air.

5. The oxygen supply device according to claim 4, wherein the threaded connection is a threaded connection Rd40 according to the standard EN 148 Part 1.

6. The oxygen supply device according to claim 1, further comprising a detection mechanism arranged in the intake nozzle, wherein the detection mechanism detects whether a filter unit is inserted and, if applicable, additionally detects which type of filter is inserted.

7. The oxygen supply device according to claim 6, wherein the detection mechanism is a mechanical pressure switch in a thread of the intake nozzle.

8. The oxygen supply device according to claim 6, wherein the detection mechanism is connected to a control unit and the control unit controls an output of the oxygen concentrator and, if applicable, is connected to an information unit which provides a user with information on a presence of a filter unit and/or the changed output of the oxygen concentrator.

9. The oxygen supply device according to claim 1, wherein the oxygen concentrator is a pressure swing adsorption (PSA) system or a vacuum pressure swing adsorption (VPSA) system and has one or a plurality of adsorber columns.

10. The oxygen supply device according to claim 1, wherein the oxygen supply device has one or a plurality of sensors in the housing, which are connected to the gas line system.

11. An oxygen supply system, having an oxygen supply device according to claim 1 and a vehicle-compatible device holder with a securing device.
